(19) United States
(12) Patent Application Publication
(10) Pub. No.: US 2003/0092665 A1
(43) Pub. Date: May 15, 2003

(54) Methods and compositions for administering DNA to mucosal surfaces

(76) Inventor: Richard W. Compans, Atlanta, GA (US)

Correspondence Address: GREENLEE WINNER AND SULLIVAN P C, 5370 MANHATTAN CIRCLE, SUITE 201, BOULDER, CO, 80303, US

(21) Appl. No.: 10/243,472

(22) Filed: Sep. 13, 2002

Related U.S. Application Data
(63) Continuation of application No. 09/430,612, filed on Oct. 29, 1999.
Application No. 09/430,612 is a continuation of application No. PCT/US98/08704, filed on Apr. 30, 1998.
(60) Provisional application No. 60/045,108, filed on Apr. 30, 1997.

(51) Int. Cl. A61K 48/00; A61K 9/127; C12N 15/86; C12N 15/88

(52) U.S. Cl. 514/44; 424/450; 435/456; 435/458

(57) ABSTRACT

The present invention provides an economical, nontraumatic and surprisingly effective method for producing a desired protein (especially a biologically active protein) or an antigen using nucleic acid molecules encoding the protein or antigen delivered to a mucosal surface of the animal or human. Expression of the antigen coding sequence exposes the immune system of the animal or human to the antigen with the result that an immune response results, especially an antigen-specific IgA response at mucosal surfaces. Desirably, the nucleic acid molecules are formulated with a bioadhesive agent in an amount sufficient to improve adherence to cells at the mucosal surfaces, thereby improving uptake of the nucleic acid molecules.

### CROSS REFERENCE TO RELATED APPLICATIONS

This application is a continuation application of U.S. patent application Ser. No. 09/430,612, filed Oct. 29, 1999, which is a continuation of PCT/US98/08704, which was filed Apr. 30, 1998 and designates the United States, which application claims priority from U.S. Provisional Application No. 60/045,108, filed Apr. 30, 1997.

### ACKNOWLEDGMENT OF FEDERAL RESEARCH SUPPORT

This invention was made, at least in part, with funding from the National Institutes of Health. Accordingly, the United States Government has certain rights in this invention.

### BACKGROUND OF THE INVENTION

The field of the present invention is the area of administering DNA to mucosal surfaces for uptake and expression, and as it relates to immunizations and to gene therapy.

There is a longfelt need in the art for efficient administration, uptake and expression of coding sequences in vivo. One important application of the technology of the present invention is the use of recombinant DNA molecules (e.g., expression vectors) for immunization or for gene therapy. For immunizations, it is especially important to develop effective methods and compositions for the development of immunity at mucosal surfaces, especially for protection of the animal or human from pathogens including, without limitation, viruses, bacteria, fungi and parasites.

### SUMMARY OF THE INVENTION

It is an object of the present invention to provide methods for the efficient administration and uptake (with subsequent expression) of heterologous nucleic acid molecules, especially recombinant DNA molecules which contain a particular sequence to be in the cell into which the nucleic acid molecule has been incorporated. The sequence is operably linked to transcription regulatory sequences so that transcription occurs in the cell into which the nucleic acid molecule has been incorporated. That sequence can lead to the production of an antisense molecule which produces a desired effect within the cell, or the sequence can encode a protein such as an antigen against which a specific immune response is desired, a cytokine, a functional protein which is to replace a nonfunctional protein in a host cell which is deficient in that function, or other protein of interest. Where the protein expressed by the administered nucleic acid molecule is an antigen, the animal or human into which it has been administered raises a specific immune response to that protein, and where that protein so expressed is an antigen of a pathogen or parasite, protective immunity to that pathogen or parasite results. The nucleic acid molecule can be a plasmid, a viral genome, viral vector, recombinant DNA molecule, oligonucleotide in the sense or antisense orientation, or DNA prepared from a particular target cell.

A desirable method of administration of a nucleic acid molecule as set forth above is the application of a composition comprising said nucleic acid molecule to at least one mucosal surface of the animal or human in which protection is sought or at which the expression of a biologically active nucleic acid or protein is desired. That mucosal surface can be at any site within the respiratory system, desirably intranasal or pharyngeal surfaces, or it can be in the oral cavity, the gastrointestinal system or urogenital system. Desirably, the nucleic acid-containing composition further comprises a bioadhesive agent which improves adherence to and uptake by the mucosal epithelium at or near the site of application. Suitable bioadhesive agents, also termed mucoadhesive agents herein, impart increased viscosity to the nucleic acid-containing composition, improve adherence of the composition to mucosal surfaces or to the mucin coating a mucosal surface, prevent dilution of the nucleic acid-containing compositions on the mucosal surface, protect the nucleic acid from attack by nucleases, and/or improve uptake, and can be one of the compounds in Table 1 hereinbelow. The mucoadhesive polymer can also or alternatively improve uptake by effectively dehydrating (or reducing hydration) of the mucosal epithelium and thereby increase uptake of the nucleic acid molecule. Suitable mucoadhesive polymers can bind to the mucins by hydrophobic interactions, van der Waals forces, interaction of charge groups, polymer admixing and linear chain association, binding of specific residues or interaction of ligands and receptors. Where the composition for mucosal administration contains nucleic acid molecules encoding an antigen, the composition can further contain at least one adjuvant capable of enhancing the immune response to the antigenic expression product of the nucleic acid molecule.

TABLE 1
EXAMPLES OF COMPOUNDS THAT ARE CONSIDERED TO BE
MUCOADHESIVE POLYMERS

Poly (acrylic acid)
Tragacanth
Poly (methyl vinylether co-maleic anhydride)
Poly (ethylene oxide)
Methylcellulose
Sodium alginate
Hydroxypropylmethyl cellulose
Carboxymethylcellulose
Methylethyl cellulose
Hydroxypropylcellulose
Karya gum
Soluble starch
Gelatin
Pectin
Poly (vinyl pyrrolidine)
Poly (ethylene glycol)
Poly (vinyl alcohol)
Poly (hydroxyethylmethylacrylate)
Carbopol
Polycarbophil
Hydroxyethylcellulose

A nucleic acid administered can encode a viral antigen, especially of a virus which infects at a mucosal surface, including but not limited to human, simian or bovine immunodeficiency virus, feline infectious peritonitis virus, influenza virus, parainfluenza virus, rhinovirus, polio virus, among others. As specifically exemplified herein, the protective antigen expressed via the recombinant (genetically engineered) nucleic acid molecule was derived from influenza virus, and the mucosal administration of this recombinant nucleic acid molecule (DNA as exemplified) led to protective immunity to infection by the corresponding influenza virus in the animal (mouse) which had been immunized with the nucleic acid-containing composition.

A biologically active nucleic acid can be an antisense molecule, which when expressed in the tissue into which the nucleic acid molecule (especially a recombinant DNA molecule) has been introduced, inhibits the expression of a gene in the cell, it could be a sense nucleic acid molecule which stimulates the expression of a gene of interest. A protein expressed from the introduced nucleic acid molecule can be a cytokine to nonspecifically modulate the immune system or it can be an active protein such as a hormone or a prohormone which can be converted in vivo to the active form (e.g., insulin or proinsulin). Additionally, the protein expressed can be an antigen of a pathogen, desirably one which infects via mucosal surfaces.

The present infection provides an improved method for the production of secretory antibodies (e.g., IgA) at mucosal surfaces, thus improving the development of protective immunity at mucosal surfaces and effectively increasing the resistance of the treated animal or human to infection by the relevant pathogen or parasite.

The immunogenic compositions of the present invention generally include a physiologically acceptable carrier, and can further include component(s) which stimulate an immune response and/or enhance persistence at or near the site of administration. Where natural infection occurs at mucosal surfaces and where protective immunity is desired, administration of the immunogenic composition is accomplished by a route which favors the development of protective immunity at mucosal surfaces; a preferred route of administration is intranasal, oral, vaginal, rectal or respiratory aerosol administration of the immunogenic composition. Compounds which stimulate the development of mucosal immunity, e.g., the nontoxic cholera toxin B subunit, among others, can be added to the nucleic acid-containing composition.

### BRIEF DESCRIPTION OF THE DRAWINGS

FIGS. 1A-1B illustrate antibody responses in serum and saliva after immunization with HA DNA alone or HA DNA and liposomes (Dosper). Four groups of mice were immunized with HA DNA alone or HA DNA/Dosper mixture either intranasally or orally. The DNA/Dosper mixture was made by mixing 20 μg HA DNA with Dosper at 1:2 ratio (w/w). The mice were immunized twice at 3 week intervals, 20 μg DNA for each dose. Blood and saliva samples were collected before and three weeks after each immunization. Virus-specific total antibody in serum and IgA in saliva were measured by ELISA. FIG. 1A shows total antibody responses in serum (sera were diluted 100 folds in PBS). FIG. 1B shows IgA responses in saliva.

FIGS. 2A-2B illustrate antibody responses in serum and saliva after immunization with HA (hemagglutinin) DNA or HA DNA and polymer zyn1000. Four groups of mice were immunized with HA DNA or an HA DNA/zyn1000 mixture either intranasally or orally. The DNA/zyn1000 mixture was made by mixing 20 μg HA DNA with zyn1000 solution to make the final concentration of zyn1000 2%. The mice were immunized twice at 3 week intervals, 20 μg DNA for each dose. Blood and saliva samples were collected before and three weeks after each immunization. Virus-specific total antibody in serum and IgA in saliva were measured by ELISA. FIG. 2A shows total antibody response in serum (sera were diluted 100 folds in PBS). FIG. 2B shows IgA response in saliva.

FIGS. 3A-3B display results for the detection of SIV-specific Ig in serum samples of mice mucosally immunized with SIV env genes. In FIG. 3A, mice were orally immunized with DNA plus CT as described in the hereinbelow: Group A with pRE239-RE(t) which encodes a truncated Env protein, and Group B with pRE239-RE which encodes a full length Env protein. Sera were obtained from individual mice of each group: preimmune, post oral inoculation #1 (8 weeks), post oral inoculation #2 (15 weeks). In FIG. 3B, Group A mice were injected intramuscularly with pRE239-RE(t). Group B mice were inoculated intranasally with LT and pRE239-RE(t). Samples were obtained from individual mice of each group pre-immune, post immunization #1 (5 weeks), and post immunization #2 (10 weeks). The reactivities of these samples (1:200 dilution) with SIV antigens were determined by ELISA. The mean results are shown for each group.

### DETAILED DESCRIPTION OF THE INVENTION

The present invention provides methods and compositions for delivery of isolated naturally occurring or engineered nucleic acid molecules to mucosal surfaces for uptake and subsequent expression of a sequence operably linked to transcription control sequences appropriate to the cell type and species into which it is introduced. The sequence expressed can encode an antigen from a virus, bacterium, fungus, protozoan or other pathogen, especially one which infects mucosal surfaces, or it can be an antigen from a parasite. Alternatively, it could be a tumor antigen. Administration of an antigen coding sequence confers protection against infection by the pathogen or parasite (or protects from establishment of the tumor). A further alternative is that a biologically active protein or prohormone or peptide can be expressed at the mucosal surface where the engineered nucleic acid molecule has been incorporated to supplement or replace an insufficiency or deficiency at that site, or its expression can serve to improve the immune response to an antigen expressed from an additional coding sequence, such as that for an immunostimulatory cytokine, administered therewith.

Most infectious agents of humans initiate infection at the mucosal surfaces of the respiratory, gastrointestinal or urogenital tracts. In some cases, the infectious agent is able to penetrate through the epithelial cell layers and spread to underlying tissues, resulting in a systemic infection. With other agents, the infection is limited to the cells of the epithelial surface. Because of the central role of mucosal epithelial cells in the initiation of both types of infection, the ability to introduce specific DNA molecules into such cells is of great current interest. Such DNA genes can encode molecules that have antimicrobial properties or vaccine epitopes that would elicit specific immune responses at mucosal surfaces. Mucosal immune responses represent the primary defense against infection at these sites. In addition to the great potential for control of microbial infections, the ability to introduce DNA plasmids efficiently at mucosal surfaces may also have therapeutic applications for control of other diseases, such as autoimmune disorders.

Expression systems similar to that specifically exemplified for influenza virus can be made for other viral antigens, including antigens from measles, mumps, parainfluenza, paramyxovirus, HIV, human T-cell leukemia type I virus, feline immunodeficiency virus, feline leukemia virus, equine infectious anemia virus, bovine immunodeficiency virus and bovine leukemia virus, among others, without the expense of undue experimentation, using nucleotide sequence information and nucleic acid vectors readily accessible to the art, taken together with the guidance and teachings provided herein. A large number of biologically active proteins, peptides and prohormones and the nucleotide sequences encoding them are also known to the art. Examples are presented in WO 95/20660, but many others are readily accessible, and engineered nucleic acid molecules can be prepared without the expense of undue experimentation and incorporated into compositions for administration to mucosal surfaces and expression in mucosal epithelial cells.

A vector is a genetic unit (or replicon) to which or into which other DNA segments can be incorporated to effect replication, and optionally, expression of the attached segment. Examples include plasmids, cosmids, viruses, chromosomes and minichromes. Also within the scope of the term vector as used herein are RNA molecules, such as RNA viral genomes which have been engineered for the expression of a sequence of interest, such as an antisense or a sense RNA or a coding sequence, in a cell into which the molecule has been introduced.

Examples of replicating viruses and replicating viral vectors which have been engineered to express foreign genes include vaccinia and herpes viruses. Vaccinia virus recombinants have been used to introduce genes encoding viral antigens into cells at mucosal surfaces. Intranasal or intraduodenal administration of such vaccinia recombinants has been found to induce specific antiviral immune responses [Meitin et al. (1991) Vaccine 9, 751-756; Meitin et al. (1994) Proc. Natl. Acad. Sci. USA 91, 11187-11191]. The expression of genes is of short duration because the virus is cytopathic and is eliminated by host defense mechanisms. Importantly, a strong immune response is induced against the antigens of vaccinia virus, which would be expected to interfere with subsequent application of such vectors [Kantele et al. (1991) Vaccine 9, 428]. The use of a live virus vector also raises safety considerations, since vaccinia virus can cause severe generalized infection in immunosuppressed individuals.

Replication-competent herpes viruses have also been investigated as a possible system for gene delivery. Mutants of herpes simplex virus type I have been obtained which are restricted for growth in non-dividing cells but are able to replicate in tumor cells. Such viruses are being investigated as possible agents for tumor therapy [Martuza et al. (1991) Science 252, 854-856]. While this approach has potential for some applications, it would not be suitable as a generalized approach for gene delivery to mucosal tissues for the same reasons as those indicated above with vaccinia virus: safety considerations, and the host immune response to the vector.

A number of vectors are based on viruses which have been modified to delete certain genes which are essential for their replication; such replication-defective viruses have been applied in gene therapy studies, particularly in lymphoid cells.

Retrovirus vectors offer the potential advantages of a broad host range, including the possibility to target infection to specific cell types by the use of genetically engineered envelope glycoproteins [Kasahara et al. (1994) Science 266, 1373-1376; Valsesia-Wittmann et al. (1994) J. Virol. 68, 4609-4619]. However, the virus titers obtained are comparatively low and may be difficult to quantitate. More importantly, integration of retrovirus genomes is a prerequisite for gene expression and occurs only in dividing cells. This limits the applicability of such vectors for use in delivering genes to many differentiated cell types in which low rates of cell division are observed.

Adenovirus vectors can be propagated to high titers, and the virus exhibits tropism for epithelial cells of the respiratory and/or enteric tracts. Gene expression does not require integration and thus, will occur in non-dividing cell types. Although host immune responses may interfere with repeated application of a single virus recombinant, multiple serotypes are available to circumvent this problem. However, administration of recombinant adenovirus vectors at high titers has been observed to induce a prominent inflammatory responses in the respiratory tract of baboons [Simon et al. (1993) Hum. Gene Ther. 4, 771-780]. Previous studies also demonstrated that high doses of adenovirus can induce an inflammatory response in mice even though the virus is unable to replicate in this species [Ginsberg et al. (1991) Proc. Natl. Acad. Sci. USA 88, 1651-1655]. It may be possible to construct improved adenovirus vectors with reduced capacity for inducing inflammation by deletion of additional genes, but such viruses may prove difficult to propagate. It is also possible that the protein components of the virus inoculum itself may retain the potential to induce significant immune responses.

Adeno associated virus (AAV) has been developed as a vector [Muzyczka, M. (1992) Curr. Top. Microbiol. Immunol. 158, 97-129]. AAV has the advantages of broad host range, lack of induction of any known human diseases, and the ability to infect non-dividing human cells [Kaplitt et al. (1994) Nature Genet. 8, 148-153]. Although a helper virus such as adenovirus is generally required for its replication, the AAV DNA can integrate into the host cell genome in the absence of a helper virus. Many of these features of AAV are attractive for potential use as vectors for gene therapy for human diseases. One potential disadvantage, however, is the limited capacity for packaging foreign genes into the recombinant virus particles. Little information is available on the immune responses generated by administration of AAV to experimental animals. However, it seems likely that host immune responses could interfere with multiple applications of such vectors.

Recombinant poliovirus is also being developed as a vector system for expression of foreign genes [Choi et al. (1991) J. Virol. 65, 2875-2883; Percy et al. (1992) J. Virol. 66, 5040-5046; Andino et al. (1993) EMBO J. 12, 3587-3598; Porter et al. (1993) J. Virol. 67, 3712-3719; Porter et al. (1995) J. Virol. 69, 1548-1555; Mattion et al. (1994) J. Virol. 68, 3925-3933]. Proteins such as the HIV-1 Gag proteins have been expressed and were found to induce immune responses in experimental animals. Limitations with this system include packaging constraints as well as a high level of pre-existing immunity to poliovirus in the human population. In particular, recipients of live attenuated oral polio vaccine exhibit long-lived mucosal immune responses to the virus, which responses may interfere with the successful administration of a recombinant poliovirus expressing foreign genes.

An alternative approach for gene delivery is the direct administration of a DNA plasmid expression vector. Such plasmid vectors were reported to be more efficient than viral vectors for gene transfer to muscle tissue [Davis et al. (1993) Hum. Gene Ther. 4, 733-740]. The potential to deliver DNA to mucosal surfaces by intranasal administration has also been reported [Fynan et al. (1993) Proc. Natl. Acad. Sci. USA 90, 11478-11482]. However, little information has ben obtained concerning the most effective route for delivery of plasmid DNAs to various mucosal surfaces.

Intramuscular injection of DNA expression vectors in mice or primates results in the uptake of DNA and the expression of the encoded proteins by the muscle cells [Acsadi et al. (1991) Nature 352, 815; Wolff et al. (1990) Science 247, 1465-1468]. DNA plasmids have also been utilized for direct introduction of genes into other tissues [Ono et al. (1990) Neurosci. Lett. 117, 259-263; Jiao et al. (1992) Exp. Neurol. 115, 400-413; Davidson, B. L., and Roessler, B. J. (1994) Neurosci. Lett. 167, 5-10]. Plasmids were found to be maintained episomally without replication [Wolff et al. (1990) Science 247, 1465-1468], and expression of the encoded proteins was observed to persist for extended time periods [Hansen et al. (1991) FEBS Lett. 290, 73; Kitsis et al. (1991) Proc. Natl. Acad. Sci. USA 88, 4138; Lin et al. (1990) Circulation 82, 2217; Wolff et al. (1992) Hum. Mol Genet. 1, 363]. DNAs encoding various genes have been used to induce both humoral and cellular immune responses to the expressed proteins. Direct immunization with DNA offers several advantages over protein subunit vaccines. Preparation of plasmid DNA is simple and inexpensive. Furthermore, the expressed proteins have the ability to induce humoral as well as cellular immune responses since the proteins are produced intracellularly and are introduced into the antigen-processing pathway that results in the generation of virus-specific cytotoxic lymphocytes (CTL). There are no packaging constraints as are observed in several viral vector systems. The long term expression of proteins may provide long-lived humoral and cell-mediated immune responses through constant stimulation of B and T cells.

Introduction of foreign DNA into animals has been primarily achieved by intramuscular injection or gene gun delivery [Tang et al. (1992) Nature 356, 152-154; Ulmer et al. (1993) Science 259, 1745-1749; Wang et al. (1993) Proc. Natl. Acad. Sci. USA 90, 4156-4160; Haynes et al. (1994) Vaccines 94, Cold Spring Harbor Laboratory Press, 65-70]. Although protective immune responses have been also demonstrated in chickens with DNA encoding influenza HA after intravenous, intraperitoneal or subcutaneous administration, only low to undetectable levels of antibody responses were observed in these animals [Robinson et al. (1993) Vaccine 11, 957-960]. The effect of mucosal administration of DNA has not been extensively investigated, and uptake of DNA from epithelial surfaces may not be as effective as direct injection of DNA into muscle cells. However, it is possible to enhance the uptake of DNA by specific delivery mechanisms.

Recently various proteins, including antigens of influenza virus, simian immunodeficiency virus, and human parainfluenza virus type 3 have been incorporated into biodegradable microspheres to be used in mucosal immunization [Eldridge et al. (1991) Mol. Imunol. 28, 287-294; Marx et al. (1993) Science 260, 1323-1327; Moldoveanu et al. (1993) J. Inf. Dis. 167, 84-90; Ray et al. (1993) J. Infect. Dis. 167, 752-755; Offit et al. (1994) Virology 203, 134-143]. Microencapsulation involves the incorporation of a bioactive agent in a protective material which is polymeric in nature. Most studies have utilized DL-lactide and glycolide copolymers (DL-PLG); the components are biocompatible esters that biodegrade in vivo into the normal metabolites lactic and glycolic acids. The potential advantages of this system include the protective effect of microencapsulation against degradation by digestive enzymes when administered orally, the ability of certain sizes of microspheres to be taken up selectively by cells in lymphoid tissues, and the flexibility in formulating the polymer to modulate the time course of release [Mestecky et al. (1994) J. Control. Rel. 28, 131-141]. Although DNA plasmids are quite stable, microencapsulation of DNA provides advantages for mucosal delivery, including the possible enhancement of uptake at mucosal surfaces.

Liposomes containing nucleic acid molecules have been used in animals and in humans for mucosal immunization and have improved immunogenicity when compared to free antigen [Wachsmann et al. (1985) Immunology 54, 189-194; Thapar et al. (1991) Vaccine 9, 129; Gupta et al. (1993) Vaccine 11, 293-306; Michalek et al. (1992) Adv. Exp. Med. Biol. 327, 191-198].

A coding sequence is a nucleotide sequence that is transcribed into mRNA and translated into protein, in vivo or in vitro.

Regulatory sequences are nucleotide sequences which control transcription and/or translation of the coding sequences which they flank. Where a coding sequence is to be expressed in the animal or human into which the vector of the present invention has been introduced, it is understood that all necessary regulatory signals for translation as well as transcription are provided.

Processing sites are described in terms of nucleotide or amino acid sequences (in context of a coding sequence or a polypeptide). A processing site in a polypeptide or nascent peptide is where proteolytic cleavage occurs, where glycosylation is incorporated or where lipid groups (such as myristoylation) occurs. Proteolytic processing sites are where proteases act.

Retroviral envelope proteins, and preferably further comprising Gag proteins from the same retroviruses, can be readily produced without the expense of undue experimentation by the ordinary skilled artisan using the teachings of the present application taken with baculovirus vectors and what is well known to and readily accessible to the art. Sequence information is known for feline leukemia virus [see, e.g., Boomer et al. (1994) Virology 204, 805-810; Rohn et al. (1994) J. Virol. 68, 2458-2467, and references cited in both of the foregoing], feline immunodeficiency virus [see, e.g., Pancino et al. (1993) Virology 206, 796-806, and references cited therein], bovine immunodeficiency virus [see, e.g., Chen et al. (1994) J. Virol. Methods 47, 331-343, and references cited therein], human T-cell leukemia virus type I [see, e.g., Wang et al. (1993) AIDS Res. Hum. Retroviruses 9, 849-860; Pique et al. (1990) EMBO J. 9, 4243-4248; Vile et al. (1991) Virology 180, 420-424, and references cited in the foregoing references], bovine leukemia virus [see, e.g., Oroszlan et al. (1984) Princess Takamatsu Symp. 15, 147-157; Sagata and Ikawa (1984) Princess Takamatsu Symp. 15, 229-240]; and equine infectious anemia virus [see, e.g., Cunningham et al. (1993) Gene 124, 93-98; Schiltz et al. (1992) J. Virol. 66, 3455-3465; Ball et al. (1992) Virology 165, 601-605; Rushlow et al. (1986) Virology 155, 309-321; and references cited in the foregoing].

In another embodiment, polyclonal and/or monoclonal antibodies capable of specifically binding to a protein expressed from the nucleic acid molecule introduced into mucosal tissue of a human or animal are provided. The term antibody is used to refer both to a homogenous molecular entity, or a mixture such as a serum product made up of a plurality of different molecular entities. Monoclonal or polyclonal antibodies which specifically react with the virus-like particles of the present invention may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York. Also, recombinant immunoglobulins may be produced by methods known in the art, including but not limited to, the methods described in U.S. Pat. No. 4,816,567. Monoclonal antibodies with affinities of 10⁸ M⁻¹, preferably 10⁹ M⁻¹ to 10¹⁰ M⁻¹ or more, are preferred.

Antibodies specific for pathogens and parasites and env proteins of retroviruses are useful, for example, as probes for screening DNA expression libraries or for detecting the presence of the cognate retrovirus in a test sample. Frequently, the polypeptides and antibodies are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. United States Patents describing the use of such labels include but are not limited to U.S. Pat. Nos. 3,817,837; 3,850,752; 3,939,350; 3,996,345; 4,277,437; 4,275,149; and 4,366,241.

Antibodies specific for antigens, including but not limited to, proteins of pathogens (including viruses) or parasites are useful in treating animals, including humans, suffering from the cognate retroviral disease. Similarly, mucosal antibodies (e.g., IgA antibodies) specific for the pathogen antigen are useful in preventing infection of the immunized individual by the cognate pathogen to whom the individual is exposed. Such antibodies can be obtained by the methods described above, where DNA (or RNA) molecules encoding the antigen are administered to mucosal surfaces.

Compositions and immunogenic preparations, including vaccine compositions, comprising the nucleic acids designed for the expression of a functional RNA or protein in the cell into which they have been introduced, especially at a mucosal surface, of the present invention and capable of inducing protective immunity in a suitably treated animal or human and a suitable carrier therefor are provided. Immunogenic compositions are those which result in specific antibody production or in cellular immunity when injected into a human or an animal. Such immunogenic compositions or vaccines are useful, for example, in immunizing an animal, including a human, against infection and/or damage caused by bacteria, mycoplasmas, protozoans, and other parasites, influenza viruses, parainfluenza viruses, paramyxoviruses, retroviruses, including but not limited to, HIV, human T-cell leukemia virus (HTLV) type I, SIV, FIV, bovine immunodeficiency virus, bovine leukemia virus and equine infectious anemia virus, among others. The nucleic acid-containing preparations comprise an amount of one or more nucleic acid molecules in an amount effective for being taken up and expressed into the cells on a mucosal surface of an animal or human to which the preparation is applied. By “immunogenic amount” is meant an amount capable of eliciting the production of antibodies directed against the virus, retrovirus, bacterium, mycoplasma, protozoan or other pathogen or parasite in an animal, especially a mammal, to which the preparation has been administered. It is preferred that the route of administration and the immunogenic composition are designed to optimize the immune response on mucosal surfaces, for example, using nasal administration (via an aerosol) of the immunogenic composition.

The nucleic acid-containing composition of the present invention comprises an amount of a bioadhesive agent effective for improving the persistence of the composition to the mucosal surface to which it has been administered, and preferably includes a component which improves the uptake of the nucleic acid by the cells at the mucosal surface. A list of suitable bioadhesive agents is provided in Table 1 hereinabove. A bioadhesive agent can be incorporated into the nucleic acid-containing composition at a concentration from about 0. 1% to about 10%, from about 0.5% to about 5% or from about 0.8% to about 3%, or from about 1% to about 2%. The concentration of the nucleic acid molecule of interest can be from about 10 to about 1000 μg/ml, or from about 50 to about 250 μg/ml, or from about 90 to about 110 μg/ml.

It is understood that the dosage of the nucleic acid in the composition of the present invention depends on the animal or human to be treated and its body weight and general health status. A dosage in a mouse can be from about 20 ng to about 1000 μg nucleic acid, or from about 40 to about 200 μg/ml, or any amount there between. The dosage can be adjusted in proportion to the size and immunological status of the individual or animal.

Immunogenic carriers can be used to enhance the immunogenicity of the retrovirus-like particles, env and other components or peptides derived in sequence from any of the foregoing pathogenic agents and encoded by the recombinant or other isolated DNA or RNA delivered to a mucosal surface. Such carriers include but are not limited to proteins and polysaccharides, microspheres formulated using, e.g., a biodegradable polymer such as DL-lactide-coglycolide, liposomes, and bacterial cells and membranes. Protein carriers may be joined to the proteins or peptides derived therefrom to form fusion proteins by recombinant or synthetic means or by chemical coupling. Useful carriers and means of coupling such carriers to polypeptide antigens are known in the art. Liposomes can be prepared using any of a number of commercially available and/or well known lipids, especially cationic lipids or combinations of lipids and cationic or polycationic compounds (including without limitation, spermine, spermidine, polylysine and others, and commercially available cationic lipids as well).

The immunogenic compositions and/or vaccines may be formulated by any of the means known in the art. They can be typically prepared as injectables or as formulations for intranasal administration or for other mucosal administration (including gastrointestinal, intratracheal, intravaginal, bronchial, rectal or genital), either as liquid solutions or suspensions. Solid forms suitable for dry administration solution in, or suspension in, liquid prior to administration can also be prepared. The preparation can also, for example, be emulsified, or the protein(s)/peptide(s) and/or nucleic acid molecules encapsulated in liposomes. Where mucosal immunity is desired, the immunogenic compositions advantageously contain an adjuvant such as the nontoxic cholera toxin B subunit [see, e.g., U.S. Pat. No. 5,462,734]. Cholera toxin B subunit is commercially available, for example, from Sigma Chemical Company, St. Louis, Mo. Other suitable adjuvants are available and may be substituted therefor. It is preferred that an adjuvant for an aerosol immunogenic (or vaccine) formulation is able to bind to epithelial cells and stimulate mucosal immunity.

Among the adjuvants suitable for mucosal administration and for stimulating mucosal immunity are organometallopolymers including linear, branched or cross-linked silicones which are bonded at the ends or along the length of the polymers to the particle or its core. Such polysiloxanes can vary in molecular weight from about 400 up to about 1,000,000 daltons; the preferred length range is from about 700 to about 60,000 daltons. Suitable functionalized silicones include (trialkoxysilyl) alkyl-terminated polydialkylsiloxanes and trialkoxysilyl-terminated polydialkylsiloxanes, for example, 3-(triethyoxysilyl) propyl-terminated polydimethylsiloxane. See U.S. Pat. No. 5,571,531, incorporated by reference herein. Phosphazene polyelectrolytes can also be incorporated into immunogenic compositions for transmucosal administration (intranasal, vaginal, rectal, respiratory system by aerosol administration) [See e.g., U.S. Pat. No. 5,562,909].

The active immunogenic ingredients are often mixed with excipients or carriers which are pharmaceutically acceptable and compatible with the active ingredient. Suitable excipients include, but are not limited to, water, saline, dextrose, glycerol, ethanol, or the like and combinations thereof. The concentration of the administered antigen-encoding nucleic acid molecule in injectable, aerosol or nasal formulations is usually in the range of 0.05 to 5 mg/ml. Similar dosages can be administered to other mucosal surfaces.

In addition, if desired, the vaccines can contain or encode minor amounts of auxiliary substances such as wetting or emulsifying agents, pH buffering agents, and/or adjuvants which enhance the effectiveness of the vaccine such as cytokines. Examples of adjuvants which may be effective include, but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of an adjuvant can be determined by measuring the amount of antibodies (especially IgA, IgM or IgG) directed against the immunogen resulting from administration of the immunogen in vaccines which comprise the adjuvant in question. Such additional formulations and modes of administration as are known in the art can also be used.

Pharmaceutically acceptable salts include, but are not limited to, the acid addition salts (formed with free amino groups of a peptide) which are formed with inorganic acids, e.g., hydrochloric acid or phosphoric acids; and organic acids, e.g., acetic, oxalic, tartaric, or maleic acid. Salts formed with the free carboxyl or phosphate groups can also be derived from inorganic bases, e.g., sodium, potassium, ammonium, calcium, or ferric hydroxides, organic bases, e.g., isopropylamine, trimethylamine, 2-ethylamino-ethanol, histidine, procaine, or cationic lipids used in the preparation of the immunogenic liposome compositions.

The immunogenic compositions or vaccines are administered in a manner compatible with the dosage formulation, and in such amount and manner as will be prophylactically and/or therapeutically effective, according to what is known to the art. Precise amounts of the active ingredient required to be administered can depend on the judgment of the physician or veterinarian and can be peculiar to each individual, but such a determination is within the skill of such a medical or veterinary practitioner.

The vaccine or other immunogenic composition can be given in a single dose; two dose schedule, for example two to eight weeks apart; or a multiple dose schedule. A multiple dose schedule is one in which a primary course of vaccination can include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the nucleic acid-containing compositions of the present invention are protected from infection by the corresponding virus, pathogen or parasite.

Where the expressed sequence of interest within the genetically engineered nucleic acid molecule is for therapy, for example, by the expression of a functional protein which is lacking or impaired in its function in the animal or human especially at or near the mucosal surface to which the nucleic acid-containing composition is administered, the coding sequence can be chosen for the desired purpose. Many coding sequences for biologically active proteins are well known to and readily accessible in the art—including without limitation, insulin and proinsulin, the pump protein which is defective in cystic fibrosis patients and functional in normal individuals and protease inhibitors which can inhibit tissue destruction in emphysema patients.

Except as noted hereafter, standard techniques for peptide synthesis, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y., Old Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein. All references and patents cited in the present application are hereby incorporated by reference in their entireties.

The foregoing discussion and the following examples illustrate but are not intended to limit the invention. The skilled artisan will understand that alternative methods and minor variations may be used to implement the invention.

### EXAMPLES

### Example 1

### Animals

Female balb/c mice were purchased from Charles River Laboratory, and mice were used between 10-12 weeks of age.

### Example 2

### Vaccine DNA

The pjw4303 HA vaccine plasmid DNA was generously provided by Dr. H. L. Robinson [Fynan, et al. (1993) Proc. Natl. Acad. Sci. USA 90, 11478-11482]. This plasmid relies on the cytomegalovirus immediate-early promoter to express the hemagglutinin (HA) gene from influenza virus A/PR/8/34 (H1).

### Example 3

### Immunization and Sampling

Two delivery systems for the DNA vaccine were used in this study. The liposome preparation Dosper was purchased from Boehringer Mannheim (Indianapolis, Ind.). For immunization, 20 μg of pjw4303 DNA was mixed with 40 μg of Dosper in HBS (Hepes-buffered saline) and incubated for 30 min. at room temperature. The other is a bioadhesive polymer (zyn1000) provided by Zynaxis Inc. (Malvern, Pa.) [Ohlsson-Willhelm et al., (1996) Mucosal immunization against influenza virus using bioadhesive polymers, p. 271-277. In A. W. H. L. E. Brown and R. G. Webster (ed.), Options for the control of Influenza III, International Conference Series, Elsevier, Cairns, North Queensland, Australia]. The DNA-polymer mixture was prepared by mixing pjw4303 HA DNA with the polymer solution at the final concentration of 2% polymer. Six groups of mice were immunized with HA DNA alone, DNA/liposome mixture or DNA/polymer mixture. The routes and adjuvants used in the immunization were: Group 1, HA DNA alone intranasally; Group 2, HA DNA alone orally; Group 3, DNA/Dosper intranasally; Group 4, DNA/Dosper orally; Group 5, DNA/polymer intranasally; Group 6, DNA/polymer orally. The mice were immunized twice at 3 week intervals, using 20 μg of DNA for each dose.

Blood and saliva samples were collected before and three weeks after each immunization. Anesthetized mice were bled from retroorbital veins to obtain blood samples. Blood is collected from the retro-orbital plexus in calibrated heparinized capillary pipettes. The blood is centrifuged, the plasma collected, heat inactivated and stored at −70° C. until assayed. Saliva was collected by aspiration from the cheek pouch after intraperitoneal injection of 2 μg of carbamylcholine chloride to stimulate flow, and 1% (v/v) 100 mM phenylmethylsulfonyl fluoride (PMSF) in isopropanol was added as a protease inhibitor. Stimulated saliva is collected with capillary tubes after injection with carbamyl-choline (1-2 μg/mouse) to examine the level of local IgA production [Moldoveanu et al. (1993) J. Inf Dis. 167, 84-90]. 2 μg each of soybean trypsin inhibitor, phenylmethylsulfonyl fluoride, sodium azide, and fetal bovine serum are added before clarification and storage at −70° C.

The following procedure is used to collect intestinal secretions. Prior to the administration of anesthetic, the mice are administered four doses of 0.5 ml lavage solution, isoosmotic for mouse gut secretions, at 15 min intervals using a feeding needle [Elson et al. (1994) Handbook of Mucosal Immunology, Academic Press, San Diego, Calif., 391-402]. Fifteen min after the last dose of lavage solution, the mice are anesthetized and 15 min later administered 0.1 mg pilocarpine by intraperitoneal injection. A discharge of intestinal contents occurs over the next 10 to 15 min and is collected into a petri dish containing 5 ml of a solution of 0.1 mg/ml soybean trypsin inhibitor in 50 mM EDTA. After collection, the material is placed in a 15 ml centrifuge tube, vortexed vigorously and centrifuged (650×g, 10 min, 4° C.) to remove solid material. The supernatant is transferred to a round-bottomed polycarbonate centrifuge tube and 30 μl of 100 mM phenylmethylsulfonyl fluoride (PMFS) is added prior to further clarification by centrifugation (27,000×g, 20 min, 4° C.). After clarification, 20 μl of 1% sodium azide is added and the solution made 1% in bovine serum to provide an alternate substrate for any remaining protease activity.

### Example 4

### Antibody Assays

Total antibodies in serum and IgA in saliva were measured by enzyme-linked immunosorbent assays (ELISA) using standard protocols in 96 well plates. The antigen used was purified A/PR/8/34 (H1 N1) influenza virus grown in chicken eggs. Horseradish peroxidase-conjugated goat anti-mouse antibodies were purchased from Southern Biotechnology Associates, Inc. (Birmingham, Ala.).

### Example 5

### Plaque Reduction Assay

A/PR/8/34 influenza virus was mixed with serum at different dilutions and incubated at room temperature for 1 hr, and standard plaque assays were performed with MDCK cells. The neutralizing antibody titer is the highest dilution that can reduce the number of the plaques by 50% or more.

We have characterized a murine model as an inexpensive and sensitive animal model for the evaluation of protective immune responses induced by mucosal administration of influenza A vaccine preparations [Novak et al. (1993) Vaccine 11, 55-60]. Using a non-mouse-adapted human influenza virus to infect unanesthetized animals intranasally, we established that the optimum dose for infection of BALB/c mice was 10⁴ plaque forming units of virus and that the optimum sampling time for measurement of virus yields in the organs of the respiratory tract was 72 h after challenge. We found that the infection was initiated in the nose and progressed by descending into the trachea and lungs over a period of days. Evaluation of protection against infection clearly showed that the tissues of the mouse respiratory tract were completely protected after administration of whole killed virus intranasally and partially protected when virus was administered subcutaneously. The degree of protection was found to be correlated with the level of virus-specific IgA antibodies in saliva.

### Example 6

### Cationic Liposomes

The DNA vector (100 g) is mixed with 20 μl of an appropriate concentration of cationic liposomes, which are effective in promoting uptake and expression of DNA in cultured cells [Rose et al. (1991) Biotechniques 10, 520-525]. In liposome-mediated DNA delivery, it has been reported that endocytosis appears to play a major role in internalization of the complex [Wrobel and Collins (1994) Biochem. Biophys. Acta 1235, 296-304]. A recent study indicates that the cationic liposomes form a bead-like structure gradually covering the DNA chain and at higher concentrations, the association leads to a complete lipid coating [Gershon et al. (1993) Biochemistry 32, 7143-7151]. A fraction of the DNA is released into the cytosol, but the majority of the internalized DNA remains in endocytic compartments and is eventually degraded. The inefficient release within the cells contributes to low expression of the encoded proteins.

Membrane-active peptides enhance liposome-mediated gene delivery. It has been previously reported that an amphipathic peptide resulted in enhanced expression in cell culture of a liposome-transfected gene [Legendre, J. Y., and Szoka, F. C., Jr. (1993) Proc. Natl. Acad. Sci. USA 90, 893-897]. Amphipathic helical peptides which we have previously demonstrated to interact with cell membranes [Srinivas et al. (1992) J. Biol. Chem. 267, 7121-7127] improve the efficiency of liposome-mediated gene delivery. Peptides corresponding to HIV-1 (WMJ strain) gp160 residues 768-788 and 826-854 are synthesized using an automated peptide synthesizer (Applied Biosystems, Foster City, Calif.) and purified by HPLC. Using standard conditions for liposome-mediated transfection of 1μg of DNA, with the addition of 1-10μg of the amphipathic peptides, following the uptake of lipid-DNA complexes in endosomes, we hypothesize that amphipathic peptides promote disruption of the endosomal membrane to facilitate release of DNA into the cell.

As another approach, fasogenic peptides derived from viral fusion proteins enhance liposome-mediated gene delivery. The N-terminal “fusion peptides” of the influenza virus HA2 protein [Wharton et al. (1989) The Influenza Viruses, Plenum Press, 153-173] and the parainfluenza virus type 3 F1 protein [Spriggs et al. (1986) Virology 152, 241-251] are synthesized and purified and used to enhance liposome-mediated vaccine gene delivery, as described above. The 23 amino acid HA2 fusion peptide, which contains acidic residues, functions to promote membrane fusion at low pH, whereas the peptide derived from the PI3 F protein promotes fusion at neutral pH. Either of these peptides can promote membrane fusion with the DNA-associated liposomes, and thus enhance liposome-mediated gene delivery.

The approaches described above for studies with cells in human respiratory organ cultures (effects of toxins, cationic polymers, mucoadhesive polymers, etc.) enhance gene delivery to the respiratory or enteric tracts. These experiments are carried out using 6-8 week old BALB/C mice. The preparations used are designed based on those giving optimal results in the organ culture studies.

The mice are administered a single dose or clustered doses of 100 μg of DNA, and a similar booster dose 30 days following primary exposure. Group sizes are arranged so that at no time point do any assay groups contain fewer than 5 mice. The antigen-specific antibodies in the sera and mucosal samples are determined by end point titration in isotype specific ELISAs, and the expression of DNA are evaluated in mucosal tissues by immunocytochemistry (see below).

1. Intranasal instillation of 20 μl without anesthesia, which results in delivery only to the upper respiratory tract [Yetter et al. (1980) Infect. Immun. 29, 654-662].

2. Intranasal instillation of 50 μl after light ether or pentobarbital anesthesia (0.06 mg/g body weight, injected intraperitoneally). This protocol results in delivery of the inoculum to the entire respiratory tract, including the lungs [Yetter et al. (1980) supra].

3. Intratracheal inoculation. Bronchopulmonary delivery to mice under sodium methohexital (Brevital, Lilly, Indianapolis, Ind.) anesthesia is via the IT instillation of the DNA in a volume of 40 μl [Eldridge et al. (1991) Mol. Immunol. 28, 287-294]. After onset of anesthesia the mice are suspended by their lower incisors from an inverted U of wire, protruding from a dissecting board maintained at a 45° angle, such that the head can be pulled over the edge of the board. The pharynx is transilluminated with the aid of a fiber optic lamp, and the fluid instilled through the shaft of a blunt tipped feeding needle, inserted through the glottis, which is attached to a Hamilton syringe with a stepped dispenser through a length of teflon tubing.

For aerosol delivery, solutions of plasmids in the selected delivery vehicles are placed in an Acorn II nebulizer (Trimedco, Atlanta, Ga.), and the mice exposed to the resulting aerosol in an exposure chamber. The delivery of expression plasmids complexed with liposomes to lung cells by aerosol has been reported [Stribling et al. (1992) Proc. Natl. Acad. Sci. USA 89, 11277-11281] . This approach is useful as a nontraumatic means for gene delivery to the respiratory tract.

Oral delivery is performed by diluting the DNA preparations at an appropriate concentration in a solution of 8 parts sterile water and 2 parts 7.5% sodium bicarbonate. Mice which have been food deprived for 12 hr are administered 0.25 ml intragastrically using a blunt tipped feeding needle [Babb et al. (1981) J. Immunol. 127, 1052].

### Example 7

### Microencapsulation of DNA

Microencapsulation of proteins has been used for mucosal delivery of protein antigens in a number of recent studies [e.g., Moldoveanu et al. (1993) J. Inf. Dis. 167, 84-90; Mestecky et al. (1994) J. Control. Rel. 28, 131-141; O'Hagan et al. (1994) Novel Delivery Systems for Oral Vaccines, Boca Raton: CRC, 175-205; Offit et al. (1994) Virology 203, 134-143]. The microspheres used were usually composed of biodegradable and biocompatible materials such as poly DL-lactide-co-glycolide (DL-PLG) copolymers with nucleic acid molecules encoding pathogen antigens incorporated within such particles during their preparation. Biodegradation, which may range from several days to months depending on the lactide-glycolide proportion, proceeds by hydrolysis of ester bonds to yield catabolizable lactic and glycolic acids. The size of such microspheres can vary; those with the size range 5-10 μm are absorbed from the gastrointestinal tract through Peyer's patches (PP) where they are retained and subsequently release the nucleic acid molecules. The incorporation of DNA into biodegradable microspheres has several advantages including protection from nucleases. As a dry powder, microspheres containing DNA or RNA are stable and with the small number of substances tested so far, results indicate that their effectiveness for vaccine nucleic acid delivery is preserved for many months.

### Example 8

### Conjugation of Plasmid DNA with Polyamines

In order to incorporate DNA into microspheres it may be useful to convert the plasmids into a more compact conformation to improve incorporation into the small size microspheres. Cationic polymers are used for DNA condensation. Plasmid DNA (20 μg) in HEPES/NaCl buffer is incubated with polyamines (spermidine and/or spermine) at concentrations ranging from 50 μM to 1 mM. After incubation at 50° C. for 2 hr [Fredericq et al (1991) J. Biomol. Str. and Dyn. 8, 847-865] samples are characterized by gradient centrifugation and electron microscopy. Similar analyses with commercially available calf thymus histones (Boehringer Mannheim) and polymers of lysine (Sigma Chemical Co., St. Louis, Mo.) are performed to choose the agent which gives the optimal DNA condensation. Initially, for every 2 μg DNA, 0.5-2 μg of polylysine is used. Concentrations and conditions are modified as necessary.

Analysis of the sedimentation properties and electron microscopic examination are used to determine the extent of DNA condensation. For initial standardization studies, [³H]-plasmid DNA is used. Briefly, bacterial transformants carrying the plasmids grown in Luria broth (LB) supplemented with methyl ³H thymidine (Amersham, 10 μCi/ml final concentration). The plasmid DNA is isolated using a maxi prep kit (Qiagen, Chatfield, Calif.) following the manufacturer's instructions. 20 μg of plasmid DNA is mixed with different concentrations of appropriate basic molecules, as described above. The mixture is layered on a continuous sucrose gradient (30%−5%) and centrifuged at 100,000 g at 4° C. for 16 hr. Labeled DNA alone is run in parallel for comparison. After centrifugation, 300 μl fractions are collected and analyzed for radioactivity. The DNA in the form of a condensed complex sediments faster because it has a compact conformation. For electron microscopic studies, samples are applied to grids, stained with a solution of uranyl acetate, and examined in a Philips CM10 electron microscope. These observations enable definition of conditions for converting plasmid DNA from an extended form into a condensed conformation, which facilitates incorporation into microspheres.

The microspheres containing the purified DNA or RNA are prepared by a solvent-evaporation process using equal molar parts of DL-lactide and glycolide as a polymer. Briefly, DNA is added into a polymer solution in an appropriate solvent. The mixture is stirred vigorously to give a uniform suspension. The mixture of polymer and DNA are then mixed with a large amount of water and the emulsion is stirred at an appropriate speed. During this evaporation period, microspheres are formed. The microspheres are isolated by filtration, washed with water and dried under vacuum. This approach has been used in our previous studies of viral antigens [Moldoveanu et al. (1993) J. Inf. Dis. 167, 84-90; Marx et al. (1993) Science 260, 1323-1327].

If necessary, in order to avoid the contact with an organic solvent, a double emulsion method [Shimamoto (1987) J. Androl. 8, S14-S16] can be used as an alternative. In this method, DNA preparations are first suspended in gelatin. The aqueous solution of DNA in gelatin is then be emulsified in the copolymer solution and used for microcapsule formation by the solvent evaporation as described above.

The resulting microsphere preparations are characterized with respect to surface morphology, core loading and size distribution. The surface morphology is examined from photomicrographs obtained by scanning electron microscopy. This confirms that a smooth surface of continuous polymeric coating has been obtained, ensuring that pinholes or cracks do not allow the DNA to leach out prematurely. The DNA content (core loading) is determined by dissolving a sample of the microspheres in an appropriate solvent, extracting the DNA, determining the amount of DNA obtained and calculating the percent incorporation by weight. The size distribution of each batch of microspheres is determined by scanning electron microscopy.

### Example 9

### Nuclease Protection Assays

The plasmid DNA encapsulated into microspheres is protected from nucleases. This is confirmed by treatment with restriction endonucleases (chosen along the sequences of the vector and HA gene) and analyzed by agarose gel electrophoresis. The DNA is extracted and analyzed by gel electrophoresis, and bands are stained with ethidium bromide and visualized under UV. Absence of cleaved fragments or nicks indicates that the plasmid DNA is protected in the microspheres. Alternately, the resistance to degradation of the DNA within the microspheres is tested by DNAse protection assays. Aliquots of DNA (10 μg) in microspheres are treated with 5U of DNAase 1 and other aliquots are untreated. Both aliquots are incubated at 37° C., and aliquots of 25 μl (containing 2.5 μg DNA) are removed after 60 min or 120 min, and the reaction is terminated by addition of 2×stop buffer (from a 6×stock containing 0.4% sucrose, 0.25% bromophenol blue in Tris EDTA buffer). DNA is extracted and analyzed on an 0.8% agarose gel. This approach allows determination that plasmid DNA in microspheres is protected form nuclease and thus gives some insight about the stability of the DNA during mucosal delivery. Protection from nucleases is particularly important for oral delivery of DNA to the intestinal tract.

The functional integrity of the plasmid DNA after undergoing the encapsulation process is also determined. A bacterial transformation assay serves as an index for analyzing the functional recovery. The plasmid DNA extracted from the microspheres is purified by ethanol precipitation, dried and resuspended in water. After quantification of the DNA, competent cells (Escherichia coli DH5 alpha, GIBCO-BRL, Gaithersburg, Md.) are mixed with re-extracted DNA (approximately 0.25 to 0.5,μg DNA), incubated on ice for 30 min, heat shocked at 42° C. for 2 min and plated on LB agar containing ampicillin (50 μg/ml final concentration). The number of transformants per μg DNA is compared with those obtained from control DNA samples not subjected to microencapsulation.

### Example 10

### Lipid Vesicles Containing the HN and F Proteins of Parainfluenza Viruses

In previous studies, parainfluenza type 3 (PI3) viral glycoproteins were reconstituted into lipid vesicles, and their properties as a subunit vaccine were determined [Ray et al. (1985) J. Infect. Dis. 152, 1219-1230; Ray et al. (1988) J. Infect. Dis. 157, 648-654]. Such reconstituted vesicles containing HN and F proteins also have potent membrane fusion activity [Hsu et al. (1979) Virology 95, 476-491]. Since these parainfluenza viruses normally infect cells of the respiratory tract, such fusogenic lipid vesicles enhance gene delivery, particularly in the respiratory tract.

For preparation of PI3 virus, confluent monolayers of LLC-MK₂ cells are infected at a multiplicity of infection of 1 pfu per cell, the virus is harvested from the culture fluid at 48 hr after infection, the culture fluid is clarified from cellular debris, and virus is pelleted by centrifugation at 143,000 g for 45 min at 4° C. Pellets are resuspended in PBS, and the virus is purified by centrifugation at 300,000 g for 1 hr at 4° C. through at 30-60% discontinuous sucrose cushion. The virus band is collected from the interface, diluted with PBS, and pelleted by high-speed centrifugation at 300,000 g for 30 min at 4° C.

For preparation of viral envelope glycoproteins, purified virions are suspended in 0.1 M Tris-HCl and 0.1 M NaCl, pH 7.6, containing 2% octylglucoside and allowed to stand at room temperature (˜23 ° C.) for 30 min. The insoluble nucleocapsid portion is removed by high-speed centrifugation at 300,000 g for 30 min at 4° C. The supernatant, containing detergent-soluble envelope constituents, is dialyzed against three changes of 0.01 M Tris-HCl and 0.01 M NaCl, pH 7.6, for 48 hr at 4° C. The envelope proteins together with the endogenous viral lipids are reconstituted into vesicles upon removal of the detergent by dialysis [Ray et al. (1985) J. Infect. Dis. 152, 1219-1230].

As one approach, the liposomes containing parainfluenza viral glycoproteins are mixed with DNA plasmids prior to intranasal administration (or other delivery routes). Coadministration of liposomes with protein antigens has been reported to enhance their immunogenicity [de Haan et al. (1995) Vaccine 13, 613-616]. Coadministration of these components enhances DNA uptake.

An as alternative approach, the DNA plasmids are incorporated within the lipid vesicles which contain the viral glycoprotein. The plasmids are mixed in the aqueous phase prior to removal of octylglucoside by dialysis. Because the cytoplasmic tails of the viral envelope proteins contain multiple positively charged residues, they interact with DNA plasmids, resulting in their incorporation. For example, the PI3 F protein cytoplasmic tail contains 3 R and 4 K residues within its 23 amino acids, and only 2 acidic residues. If necessary, the coding sequence of the cytoplasmic tails could be modified to encode a stretch of lysine residues. In this case the modified F proteins are expressed in cell cultures, purified by affinity chromatography, and reconstituted into vesicles. [see, e.g., Ray et al. (1988) J. Virol. 62, 783-787]. The resulting preparations are used for intranasal or intratracheal administration.

Although almost all individuals have prior exposure to human parainfluenza virus type 3, the mucosal immune response is of short duration and reinfection readily occurs. It is therefore unlikely that pre-existing immunity in the population compromises the possible use of these viral envelope proteins as vehicles to enhance gene delivery.

### Example 11

### Detection of HA Expression

An immunohistochemistry procedure is used to evaluate HA expression in formalin-fixed, paraffin-embedded tissue sections. Sections are deparaffinized in xylene and rehydrated through graded ethanol to distilled water. Slides are washed in TBT20 (0.05 M Tris-buffered saline, pH 7.6, containing 0.5% bovine serum albumin and 0.3% Tween 20) before blocking for non-specific protein binding with 10% normal goat serum (containing 0.1 μg/l rhesus IgG to saturate Fe receptors) in TBT20. Sections are then sequentially incubated (15 minutes) in avidin and biotin solutions to block endogenous peroxidase activity prior to incubation with anti-HA rabbit serum for 1 hr at room temperature. After washing with TBT20, sections are reacted with biotinylated goal anti-rabbit IgG (Vector Labs, Burlingame, Calif.) at 1:200 dilution in .05 M Tris-buffered saline (TBS) for 30 min at room temperature, then washed in TBT20. Endogenous peroxidase activity is blocked in 0.6% H₂O₂ in absolute methanol at room temperature for 30 minutes. Slides are then incubated in ABC complex (Vector Labs) for 30 min, washed in TBT20, and developed in diaminobenzidine for 8 min. After washing, sections are counterstained in Mayer's hematoxylin. Control tissues incubated with anti-HA serum and HA transfected tissues incubated with normal rabbit serum are processed in parallel as negative controls.

To quantitate the HA expression levels in tissues, tissue samples are dissolved in lysis buffer, the proteins are separated by gel electrophoresis, and amounts estimated by a quantitative Western immunoblot procedure [Stephens et al. (1988) Gonococci and Meningococci: Epidemiolovy, Genetics, Immunochemistry Pathogenesis, Kluwer Academic Publishers, Dordrecht, The Netherlands, 827-836].

### Example 12

### Evaluation of Immune Responses

A sensitive and quantitative method for testing the efficacy of the various gene delivery systems is to measure immune responses to the epithelially expressed protein. Both humoral and cellular responses to influenza virus HA in mice are measured at various times (1, 2, 6 and 12 months) after administration of DNA. The later time points (6 and 12 months) reflect enhanced antibody response of mucosal sites and indicate persistent antigen expression.

Serum, saliva and intestinal secretions are collected and prepared for immunoassays as described in Example 3.

A great deal of evidence has been obtained for the existence of a common mucosal system in mice and humans [McGhee, J. R., and Mestecky, J. (1990) Infect. Dis. Clin. North Amer. 4, 315-341; Czerkinsky et al. (1991) Infect. Immun. 59, 996-1001]. Specific S-IgA antibodies have been detected in remote secretions (e.g., tears and milk) induced by natural intestinal exposure to antigens or oral immunization, and analyses of IgA-secreting cells from peripheral blood and mucosal tissues [Czerkinsky et al. (1991) supra] have provided strong evidence for this concept.

Enzyme-linked immunosorbent assays (ELISAs) for mouse antigen-specific IgM, IgG and IgA antibodies are carried out in 96 well assay plates coated overnight with a pre-titrated concentration of A/PR8 influenza virus in borate buffered saline (BBS). Influenza A/PR8 virus is grown in embryonated eggs and purified by gradient centrifugation [Moldoveanu et al. (1993) J. Inf. Dis. 167, 84-90]. All washing steps employ PBS containing 0.05% Tween 20 and the diluent for all samples and reagents are PBS-Tween with 1% BSA. After blocking, serial 2-fold dilutions of samples of sera or secretions are added and incubated at 25° C. for 6 hr. The antigen-binding antibodies are detected by sequential incubation with biotin-conjugated antibodies specific for the heavy chains of mouse IgM, IgG or IgA (overnight, 4° C.), horseradish peroxidase-streptavidin (2 hr, 25° C.) and the substrate 2,2′-azino-di(3-ethyl-benzthiazoline-sulfonic acid) at 0.3 mg/ml in pH 4.0 citrate buffer containing 0.0003% H₂O₂. The developed color is read after 15 min at 415 nm. The endpoint is calculated as the reciprocal of the highest sample dilution producing a signal which is 0.2 absorbance units above that of the animals prebled at the same dilution.

Antibody response is monitored by quantitating the number of HA specific plasma cells using the ELISPOT technique [Slifka et al. (1995) J. Virol. 69, 1895-1902]. Briefly, nitrocellulose-bottom 96-well Multiscreen HA filtration plates (Millipore Corp., San Francisco, Calif.) are coated with phosphate-buffered saline (PBS) containing 10 μg of purified influenza virus per ml and incubated overnight at 4° C. As an irrelevant-antigen control, wells are coated with lymphocytic choriomeningitis virus. Plates are washed once with PBS containing 0.1% Tween 20 (PBS-T) and three times with PBS, and then blocked with 200 μl of Iscove's medium containing 5% fetal calf serum for at least 1 hr to decrease the number of remaining protein-binding serum. Blocking medium is replaced with 100 μl of medium containing three-fold dilutions of cells and incubated for 4 hr at 37° C. in humid atmosphere with 6% CO₂. Plates are washed and then a 100 μl volume of biotinylated, affinity-purified horse anti-mouse immunoglobin G (IgG) (Vector Laboratories, Burlingame, Calif.) diluted 1:1,000 in PBS-T containing 1% fetal calf serum is added to each well and incubated overnight at 4° C. For measuring IgA and IgM responses the second antibody is anti-mouse IgA and anti-mouse IgM, respectively. The plates are washed four times with PBS-T, 100 μl of horseradish peroxide-conjugated avidin D (Vector Laboratories) at a concentration of 5 μl/ml in PBS-T-1%, fetal calf serum is added, and the mixture is incubated at room temperature for 1 hr. After appropriate washing, detection is carried out by adding 100 μl of horseradish peroxidase-H₂O₂. Granular red spots appear in 3 to 5 min, and the reaction is terminated by thorough rinsing with tap water. Spots are enumerated with a stereomicroscope equipped with a vertical white light. Each spot represents a plasma cell secreting antibody against the influenza virus HA.

Cell mediated immunity is assessed by monitoring both CD4+ and CD8+ T cell responses against HA after mucosal immunization with HA-encoding plasmid DNA. The following assays are employed:

Single-cell suspensions of lymphocytes are tested for cytotoxicity on uninfected and influenza virus infected MHC matched targets in a 6 hr ⁵¹Cr release assay [Lau et al. (1994) Nature 369, 648-652]. The phenotype of the effector is determined by treatment with anti-CD8+C′ and anti-CD4+C′. CTL (cytotoxic T lymphocyte) responses analyzed by a limiting dilution (LD) assay. This technique allows precise quantitation of the number of CTL specific for HA. T cell proliferative responses are checked by stimulating lymphocyte populations in vitro with purified influenza virus or influenza virus-infected syngeneic cells. At various times after culture (48, 72 and 96 hr) the cells are pulsed with [³H]thymidine and harvested 18 hr later. The proliferating cells are typed with monoclonal antibodies to CD4 and CD8. Cytokine responses are examined to determine the relative activation of TH1 and TH2 type of T cells following mucosal gene delivery. TH1 responses are monitored by determining levels of IL-2 and interferon-gamma and TH2 responses by quantitating levels of IL-4 and IL-10 [Mossman, T. R., and Coffman, R. L. (1989) Adv. Immunol. 46, 111-147]. Cytokines will be measured by bio-assays as well as ELISA. ELISA kits for measuring IL-2, interferon-gamma, IL-4 and IL-10 are available. In addition to the bio-assays and ELISA, ELISPOT assays can be used for quantitating the number of cells producing these various cytokines.

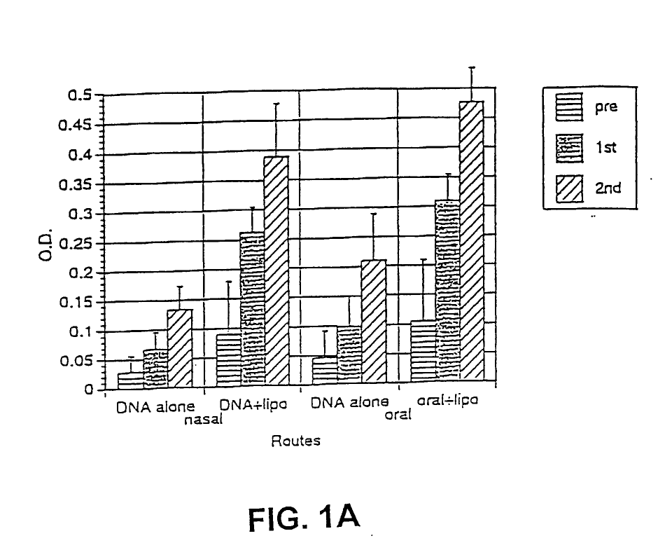

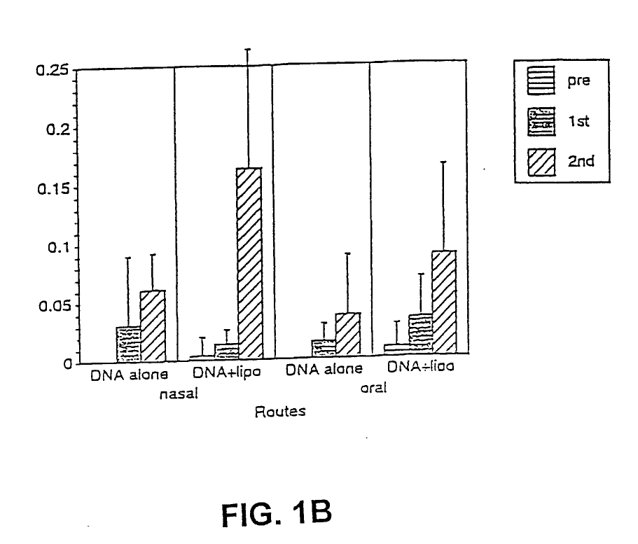

Liposomes have been used in mucosal immunization and have been found to improve immunogenicity when compared with free protein antigens [Thapar et al. (1991) Vaccine 9, 129; Michalek et al. (1992) Adv. Exp. Med. Biol. 327, 191-198; Gupta et al. (1993) Vaccine 11, 293-306; Ray et al. (1988) J. Infect. Dis. 152, 648-654]. The ability of cationic liposomes to enhance transfection of DNA into cells in culture is also well established [Ben-Ahmeida et al. (1993) Vaccine 11, 1302-1309]. To test ability of liposomes to enhance the efficiency of a DNA vaccine for delivery at mucosal surfaces, 20 μg pjw4303 HA DNA was mixed with Dosper at a ratio of 1:2 (w/w) in HBS buffer and incubated for 30 min. at room temperature. Four groups of mice were immunized twice either intranasally or orally with HA DNA alone or the DNA/Dosper mixture at 3 weeks interval. Antibody responses were measured by ELISA both in the serum and saliva (FIGS. 1A-1B). Antibody responses were induced with DNA alone, but the levels were enhanced by the use of liposome in both the intranasal and oral routes. After the first immunization, antibody responses were detected in both the intranasally and orally immunized groups with the DNA/liposome mixture. The boost gave rise to a further increase in the antibody levels, but the increases were less than 50% in both groups when compared with the antibody levels after the first immunization. (FIG. 1A).

In the saliva, only borderline IgA levels were detected in the groups immunized with DNA alone. The liposome elicited more than 2 fold increase in both groups (FIG. 1B). The first immunization of the liposome groups give rise to very low level of IgA responses. However, the IgA levels increased significantly after the boost, about 10 fold for the intranasal group and 3 fold for the oral group. The mean ELISA antibody titers of the serum and saliva are shown in Table 2. These data indicate that liposomes can be used as a mucosal delivery system for a DNA vaccine.

Another delivery system for DNA vaccine in this study is the bioadhesive polymer zyn1000 (carboxymethylcellulose, Zynaxis, Malvern, Pa.). Recently, bioadhesive polymers have generated considerable interest as a possible mode of vaccine delivery [Ohlsson-Willhelm et al., (1996) Mucosal immunization against influenza virus using bioadhesive polymers, p. 271-277. In A. W. H. L. E. Brown and R. G. Webster (ed.), Options for the control of Influenza III, International Conference Series, Elsevier, Cairns, North Queensland, Australia]. These polymers can form aqueous solutions of high viscosity, which are believed to adhere to mucosal surfaces. The rationale for the use of bioadhesive polymers for the mucosal delivery of DNA is that the interaction of such polymers with the mucin layer allows the bioadhesive and any associated DNA to bind to the mucin layer, which is associated with mucosal surfaces. This association

TABLE 2
NEUTRALIZING AND ELISA TITERS OF ANTIBODIES FROM DNA
IMMUNIZED MICE

| | | | Neutralizing | | | |
|---|---|---|---|---|---|---|
| Mouse | | Routes of | titer# | | ELISA titer* | |
| group | adjuvant | Immunization | serum | saliva | serum | saliva |
| 1 | Dosper | intranasal | 1:200 | 20 | 1:2560 | 16 |
| 2 | Dosper | oral | 1:80 | 10 | 1:2240 | 4 |
| 3 | zyn1000 | intranasal | 1:190 | 10 | 1:3200 | 8 |
| 4 | zyn1000 | oral | 1:200 | <10 | 1:1280 | 4 |
| pre† | | <50 | <10 | <50 | | <4 |

#Neutralization titer were determined as the highest dilution of serum or saliva that can reduce the number of viral plaques by 50% or more in a plaque reduction assay.
*ELISA titer were determined as the highest dilution of sera or saliva which were counted as positive readings. Absorbance were read at 405 nm and sores were judged positive when exceeding counts of negative-control by two standard deviation.
†Pre-immunization samples.

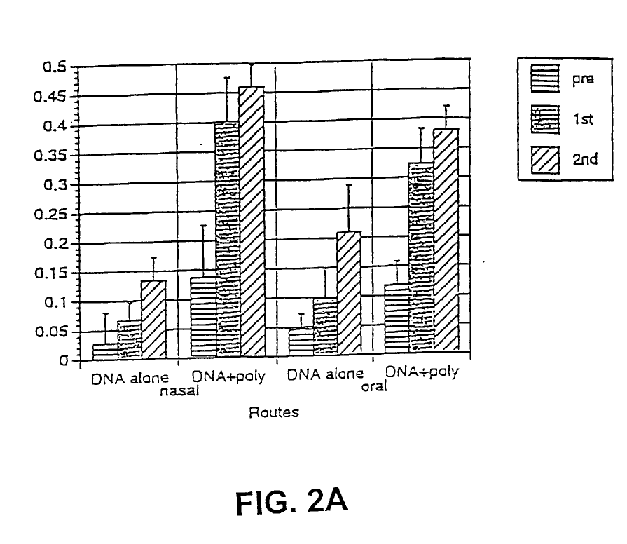

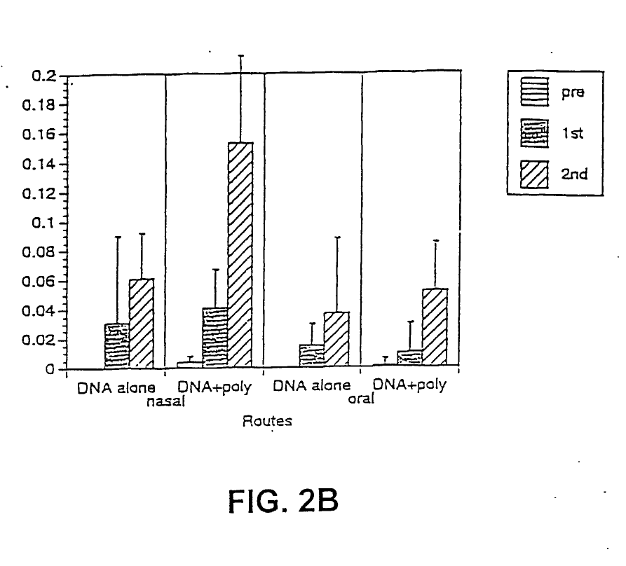

may increase the efficiency of the interaction of the DNA with the mucosal membranes by: (1) increasing the time span during which the DNA interact with the cells of the mucosal surfaces; (2) increasing the effective concentration of the DNA adjacent to mucosal membrane by separating it from large volume of fluid in the lumen; and (3) protecting the DNA from degradative enzymes by localization within the mucin layer. One study [Ohlsson-Willhelm et al., (1996) Mucosal immunization against influenza virus using bioadhesive polymers, p. 271-277. In A. W. H. L. E. Brown and R. G. Webster (ed.), Options for the control of Influenza III, International Conference Series, Elsevier, Cairns, North Queensland, Australia] has shown that the bioadhesive polymer zyn1000 enhances the immune response to a protein antigen when applied by the oral route. Here we explored the immunostimulatory effect of the polymer zyn1000 on DNA vaccine. pjw4303 HA DNA was mixed with zyn1000 solution to a final concentration of zyn1000 2%. Four groups of mice were inoculated twice with either HA DNA alone or with the DNA/zyn1000 mixture at 3 weeks intervals, 20 μg per mouse at each time. Increases of antibody responses were observed by the use polymer zyn1000 when compared with DNA alone groups. No significant increase was observed after the boost inoculation in the polymer groups, less than 20% increase was achieved in both groups (FIG. 2A).

In contrast, in the groups immunized with DNA/zyn1000 mixture, very low levels of IgA were induced in the saliva after the first immunization but more than a 3-fold increase was achieved by the second immunization (FIG. 2B). The ELISA titers of the antibodies are shown in Table 2.

To confirm that the antibody raised against DNA/Dosper and DNA/zyn1000 vaccine is functional, a plaque reduction assay was performed using live PR8 virus. As expected, the antibodies induced by these vaccines was found to neutralize the virus and block its infectivity for MDCK cells (Table 2).

Although it is relatively easy to deliver vaccines to mucosal surfaces, the efficiency of uptake of the vaccine particles is very low. Recent studies suggest that less than a fraction of one percent of the particles are taken up and translocated. Previous studies [Fynan et al. (1993) Proc. Natl. Acad. Sci. USA 90, 11478-11482; Justewicz et al. (1995) J. Virol. 69, 7712-7717; Robinson et al. Vaccine 95, Cold Spring Harbor Laboratory Press, Cold Spring Harbor, N.Y.] have evaluated the ability of DNA inoculation to raise protective immunity by mucosal routes. They found that immunization with DNA alone was less efficient than other routes (intramuscular, gene gun) and had only partial protection against live virus challenge. No significant or very low levels of antibodies in the serum and IgA in the saliva could be detected. Since M cells have cell-specific complex carbohydrates on their cell surface that could theoretically serve as receptors for mucosal vaccines, attempts have been made to improved the uptake efficiency by using lectins, but the results were not encouraging [Holmgren et al. (1996) Vaccine 14, 644-664]. In this experiment, both liposome and polymer zyn1000 were found to enhance serum antibody responses and, more importantly, IgA responses in the saliva. IgA is by far the most predominant immunoglobulin in mucosal secretions and provides the first defense line against many mucosal pathogens.

An interesting and surprising observation in our experiment is that the intranasal and oral routes induced a similar magnitude of antibody response in the serum, but IgA responses were much higher in the mice immunized intranasally than in those immunized orally. Another interesting observation is that a high serum antibody responses was achieved after the first immunization and boosting only gave a small increase. The IgA response in saliva, however, showed borderline level after the first immunization but showed a 3-10 fold increase after the boosting. This indicates that boosting is important in inducing IgA response in mucosal secretions.

DNA plasmid expression vectors expressing a full length or a truncated version of the SIVmac239 env gene have been constructed. Alternative types of plasmids: pREP7 and pREP10 (Invitrogen, Carlsbad, Calif.) with an RSV promoter, and pcDL-SRa, which contains the SV40 early promoter and R-U5′ of the long terminal repeat of HTLV-1, have been tested. Expression of SIV Env by these plasmids was evaluated in the epithelial human cell line HEp2 and in two CD4+ human suspension cell lines, H9 and Molt4. The highest level of expression was observed for constructs using the RSV promoter in all cell lines. Envelope glycoproteins expressed by these constructs also induced syncytium formation in CD4+ cell lines.

Antibody responses to the DNA vectors with the RSV promoter following mucosal immunization of mice via different routes have been examined. For gastric immunization DNA was coadministered with the enterotoxin of Vibrio cholerae (CT) which has potent mucosal adjuvant properties. For intranasal administration, DNA plasmids with Escherichia coli heat-labile toxin (LT) manufactured by the Swiss Serum and Vaccine Inst., Berne, Switzerland. LT also serves as an effective oral adjuvant. Groups of female BALB/c mice (8-12 weeks of age) were immunized with clustered inoculations, which was found to be more effective than single inoculations. Mice were given three administrations of 100 μg DNA each at 3 day intervals, and after 4 weeks the immunization regimen was repeated. Serum and saliva samples were collected after one month and analyzed by ELISA to determine SIV-specific immune responses. The results are shown in FIGS. 2A-2B.

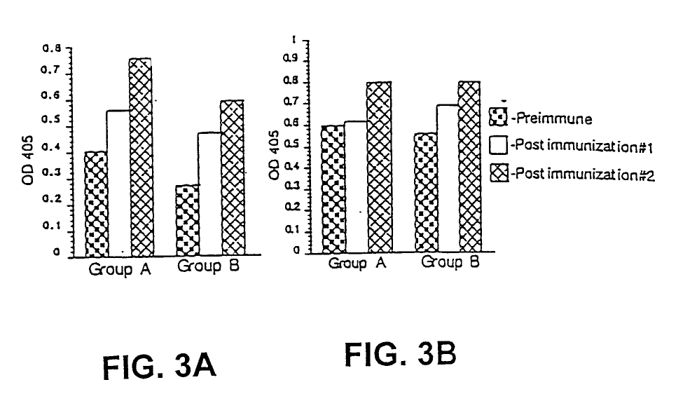

No significant difference between the immune responses of mice immunized by constructs expressing full length or truncated Env proteins was observed. From a comparison of alternative approaches for delivery of DNA with Lipofectace, with CT, or with LT, it was found that only mice immunized with CT or with LT showed immune responses. Similarly, after intramuscular injection with Lipofectace, no mice showed rises in antibody titers after two months of immunization (see FIGS. 3A-3B).

What is claimed is:

1. A method for formulating a composition for administration to a nasal or oral mucosal surface of an animal or a human, said method comprising the step of combining a nucleic acid molecule encoding all or part of a biologically active protein or said nucleic acid molecule being complementary to at least part of a messenger RNA sequence encoding a biologically active protein, with a bioadhesive polymer in an amount effective for improving retention on the mucosal surface to which the composition is administered.

2. The method according to claim 1 wherein the nucleic acid molecule is incorporated within liposomes wherein said liposomes further comprise a virus protein or virus glycoprotein promoting adherence to a mucosal cell surface.

3. The method according to claim 2 wherein the liposomes are parainfluenza virus liposomes.

4. The method according to claim 3, wherein the composition further comprises a polyamine.

5. The method according to claim 1, wherein the nucleic acid molecule encodes a biologically active protein which is not produced by the animal or human to which the composition is administered.

6. The method according to claim 1, wherein the nucleic acid molecule encodes a cytokine.

7. The method according to claim 1, wherein the nucleic molecule encodes an anti-inflammatory protein.

8. The method according to claim 1, wherein the bioadhesive polymer is poly(acrylic acid), tragacanth, polyethylene oxide, methylcellulose, carboxymethylcellulose, alginate, hydroxypropylcellulose, karya gum, starch, gelatin, pectin, polyvinyl pyrrolidine, polyethylene glycol, polyvinyl alcohol, polyhydroxymethyl acrylate, carbopol, polycarbophil or poly (methylvinylether co-maleic anhydride).

9. A method for formulating an immunogenic composition suitable for achieving an immune response in an animal or a human, wherein the immune response is specific for an antigen, said method comprising the step of combining nucleic acid molecules encoding an antigen to which an immune response is desired and a bioadhesive polymer in an amount effective for improving retention on a nasal or oral mucosal surface to which the composition is administered.

10. The method of claim 9 wherein the bioadhesive polymer is poly(acrylic acid), tragacanth, polyethylene oxide, methylcellulose, carboxymethylcellulose, alginate, hydroxypropylcellulose, karya gum, starch, gelatin, pectin, polyvinyl pyrrolidine, polyethylene glycol, polyvinyl alcohol, polyhydroxymethyl acrylate, carbopol, polycarbophil or poly (methylvinylether co-maleic anhydride).

11. The method of claim 9, wherein the antigen encoded by the DNA molecule is of viral, bacterial, fungal or protozoan origin.

12. The method of claim 9, wherein the DNA molecule encoding the antigen comprises a plasmid or a virus origin of replication.

13. The method of claim 9, wherein the DNA molecule encoding the antigen comprises a promoter operably linked to a coding sequence of an antigen, said promoter being functional in the animal or human to which the immunogenic composition comprising same is administered.

14. The method of claim 9, wherein the immunogenic composition further comprises at least one immunological adjuvant.

15. The method of claim 9, wherein said nucleic acid molecules are incorporated within parainfluenza virus liposomes.

16. The method of claim 9, wherein said composition further comprises a viral protein or viral glycoprotein which promotes adherence to a mucosal surface.

17. The method of claim 9, wherein the DNA molecules in the immunogenic composition encode an influenza hemagglutinin.
